(19) United States
(12) Patent Application Publication
(10) Pub. No.: US 2014/0283486 A1
(43) Pub. Date: Sep. 25, 2014

(54) PROCESS FOR THE PREPARATION OF FEBUXOSTAT POLYMORPHS

(71) Applicant: MYLAN LABORATORIES LTD, Hyderabad (IN)

(72) Inventors: Ramakoteswara Rao JETTI, Hyderabad (IN); Balakrishna Reddy Bhogala, Hyderabad (IN); Satish Beeravelli, Hyderabad (IN)

(73) Assignee: MYLAN LABORATORIES LTD, Hyderabad (IN)

(21) Appl. No.: 14/277,987

(22) Filed: May 15, 2014

Related U.S. Application Data
(63) Continuation of application No. PCT/IN2012/000748, filed on Nov. 12, 2012.

(30) Foreign Application Priority Data
Nov. 15, 2011 (IN) 3909/CHE/2011

(51) Int. Cl. C07D 277/56; B65B 31/00

(52) U.S. Cl. CPC C07D 277/56; B65B 31/00
USPC 53/449; 548/201

(57) ABSTRACT

The present invention relates to polymorphic forms of Febuxostat and processes for the preparation of polymorphic forms of Febuxostat.

### CROSS-REFERENCE TO RELATED APPLICATIONS

This application is a continuation of parent International Application Number PCT/IN2012/000748, which was filed Nov. 14, 2012 and claims priority to Indian Patent Application Number 3909/CHE/2011, which was filed on Nov. 15, 2011. The entire disclosure of all of the prior applications is hereby incorporated by reference herein in their entirety.

### FIELD OF THE INVENTION

The present invention relates to a novel crystalline form of Febuxostat, i.e. 2-[3-cyano-4-(2-methylpropoxy)phenyl]-4-methylthiazole-5-carboxylic acid, denominated as Form-M₁. The present invention also relates to a process for the preparation of Febuxostat Form K.

### BACKGROUND

Febuxostat, i.e. 2-(3-cyano-4-isobutyloxyphenyl)-4-methyl-5-thiazolecarboxylic acid, of Formula-I is approved by USFDA for the treatment of hyperuricemia in patients with gout under the brand name ULORIC. ULORIC is recommended at 40 mg or 80 mg once daily.

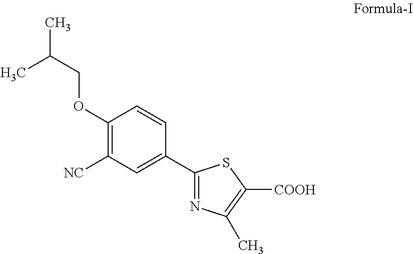 [SMILES: Cc1nc(-c2ccc(OCC(C)C)c(C#N)c2)sc1C(=O)O]

Febuxostat and its pharmaceutically acceptable salts were first disclosed in U.S. Pat. No. 5,614,520. This patent also discloses a process for the preparation of Febuxostat.

U.S. Pat. No. 6,225,474 discloses crystalline Forms A, B, C, D, G and an amorphous form of Febuxostat. This patent also discloses methods of producing crystalline Forms A, B, C, D, G and amorphous Form of Febuxostat.

Crystalline Forms F1, F2, F10, R, R1, R2, R3, R4 and R5 of Febuxostat are disclosed in U.S. Patent Application Publication No. 2010/0317702 and PCT Publication Nos. WO 2011/107911 and WO 2011/080651.

CN 101817801 discloses crystalline Form-K characterized by powder X-ray diffraction pattern having peaks at 5.65, 7.91, 11.52, 12.77, 14.29, 15.42, 16.75, 17.44, 18.14, 18.39, 20.47, 20.98, 22.23, 23.31, 23.81, 24.45, 25.89, 26.08, 28.92, 31.26 and 34.41±0.2 2θ.

The present invention provides a stable and industrially scalable crystalline form of Febuxostat and an improved process for the preparation of Febuxostat crystalline Form-K.

### SUMMARY

An object of the present invention is to provide novel crystalline Form-M₁ of Febuxostat, i.e. 2-(3-cyano-4-isobutyloxyphenyl)-4-methyl-5-thiazolecarboxylic acid. Another object of the present invention is to provide an improved process for the preparation of Febuxostat crystalline Form-K.

One aspect of the present invention provides, a process for the preparation of Febuxostat crystalline Form-M₁ comprising the steps of:
a) dissolving Febuxostat in an ester solvent,
b) spray drying the solution, and
c) isolating the crystalline Form-M₁.

Another aspect of the present invention provides an improved process for the preparation of crystalline Form-K of Febuxostat comprising the steps of:
a) dissolving Febuxostat in an ester solvent,
b) evaporating the solvent by agitated thin film dryer (ATFD), and
c) isolating Febuxostat crystalline Form-K.

Yet another aspect of the present invention provides an improved process for the preparation of crystalline Form-K of Febuxostat comprising the steps of:
a) dissolving Febuxostat in an ester solvent at 50-80° C.,
b) removing the solvent at same temperature, and
c) isolating Febuxostat crystalline Form-K.

Yet another aspect of the present invention provides an improved process for the preparation of crystalline Form-K of Febuxostat comprising the steps of:
a) dissolving Febuxostat in an ester solvent at 50-80° C.,
b) removing the solvent at same temperature,
c) adding hydrocarbon solvent, and
d) isolating Febuxostat crystalline Form-K.

### BRIEF DESCRIPTION OF THE DRAWINGS

Further objects of the present invention together with additional features contributing thereto and advantages accruing therefrom will be apparent from the following description of preferred embodiments of the invention which are shown in the accompanying figures wherein:

FIG. 1 illustrates the powder X-ray diffraction pattern of Febuxostat Form-M₁.

FIG. 2 illustrates the DSC thermogram of Febuxostat Form-M₁.

FIG. 3 illustrates the powder X-ray diffraction pattern of Febuxostat Form-K

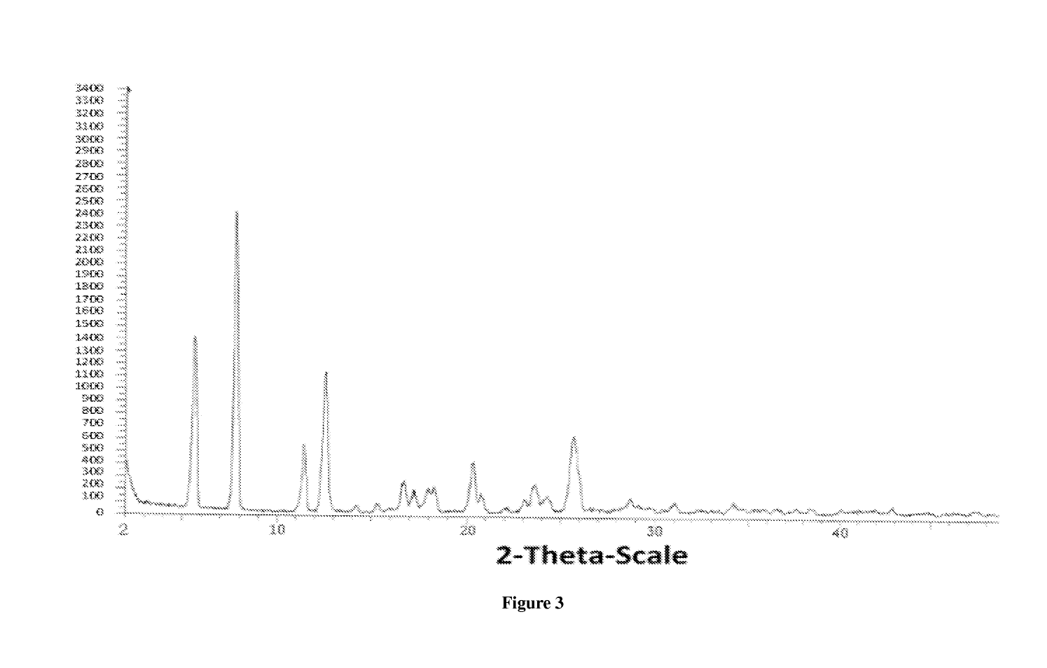

FIG. 4 illustrates the DSC thermogram of Febuxostat Form-K.

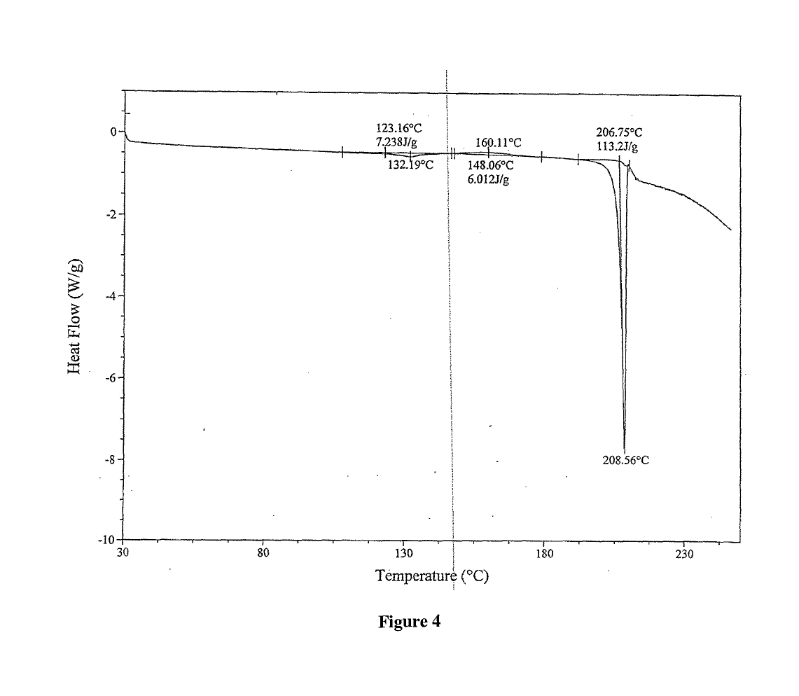

FIG. 5 illustrates the TGA thermogram of Febuxostat Form-K.

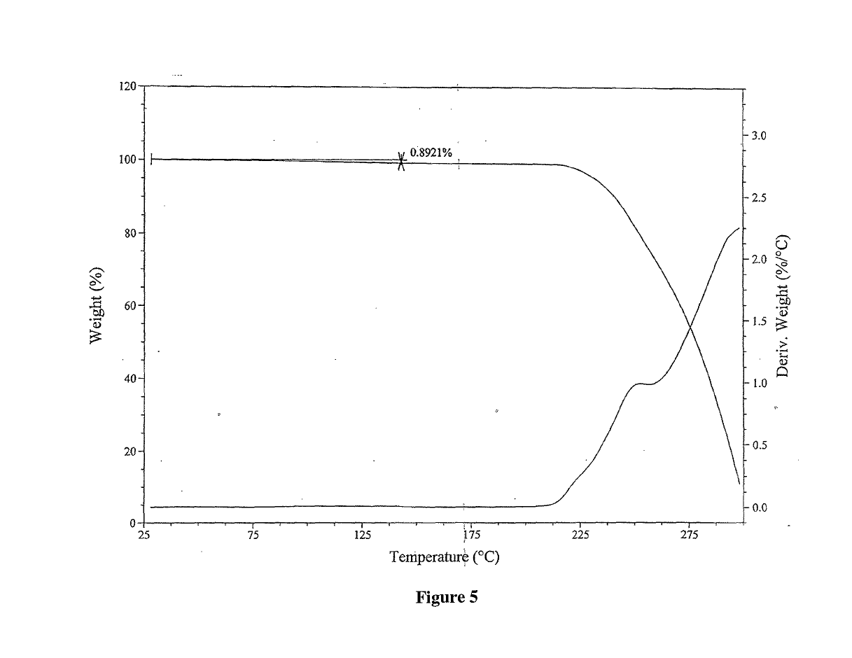

FIG. 6 illustrates the thermal ellipsoid plot of Febuxostat Form-K with atomic numbering scheme.

FIG. 7 illustrates the powder X-ray diffraction patterns of Febuxostat Form-K (Experimental and Simulated pattern).

### DETAILED DESCRIPTION

The present invention relates to novel crystalline Form-M₁ of Febuxostat, i.e. 2-(3-cyano-4-isobutyloxyphenyl)-4-methyl-5-thiazolecarboxylic acid.

### Instrumentation

### Powder X-ray Diffraction (PXRD)

The said polymorphs of the present invention are characterized by X-ray powder diffraction patterns. Thus, the X-ray diffraction patterns of said polymorphs of the invention were measured on 1) PANalytical, X'Pert PRO powder diffractometer equipped with goniometer of θ/θ configuration and X'Celerator detector and 2) Bruker AXS D8 Discover powder X-ray diffractometer equipped with a goniometer of θ/2θ configuration, Variol monochromator and Lynx-Eye detector. The Cu-anode X-ray tube was operated at 40 kV and 30 mA. The experiments were conducted over the 20 range of 2.0°-50.0°, 0.030° step size and 50 seconds step time.

Differential Scanning calorimetry (DSC)

The DSC measurements were carried out on TA Q1000 of TA instruments. The experiments were performed at a heating rate of 10.0° C./min over a temperature range of 30-300° C. purging with nitrogen at a flow rate of 50 mL/min. Standard aluminum crucibles covered by lids with three pin holes were used.

### Thermo Gravimetric Analysis (TGA)

TGA/DTA was recorded using the instrument TA Q5000 of TA instruments. The experiments were performed at a heating rate of 10.0° C./min over a temperature range of 30-300° C. purging with nitrogen at a flow rate of 25 mL/min.

An aspect of the present invention is to provide novel crystalline Form-M₁ of Febuxostat.

In one embodiment, crystalline Febuxostat Form M, is characterized by the powder X-ray diffraction having characteristic peaks at 6.13, 9.06, 12.29, 17.44 and 25.81±0.2°2θ.

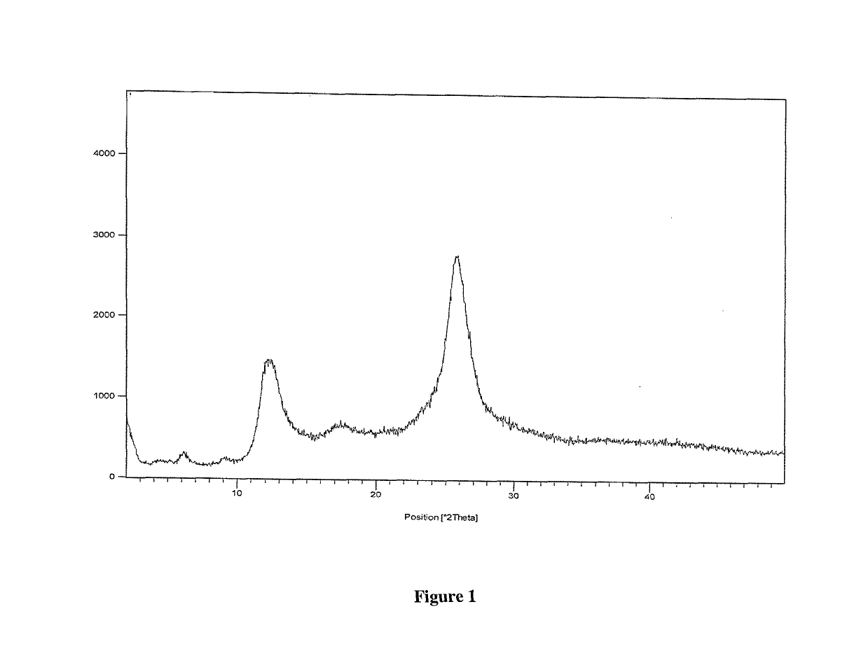

In another embodiment, crystalline Form-M, of Febuxostat is further characterized by the Powder X-ray diffraction as depicted in FIG. 1.

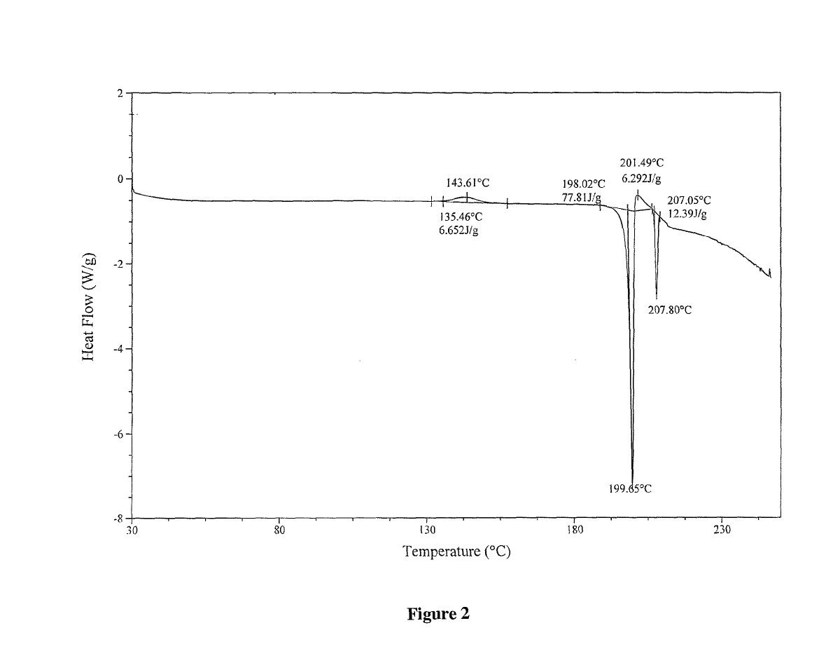

In another embodiment, crystalline Form-M₁ of Febuxostat is further characterized by the DSC thermogram as depicted in FIG. 2.

Another aspect of the present invention provides a process for the preparation of Febuxostat crystalline Form-M, comprising the steps of:
a) dissolving Febuxostat in an ester solvent,
b) spray drying the clear solution, and
c) isolating the crystalline Form-M₁.

In one embodiment, the ester solvent used herein is Ethyl acetate.

In another embodiment, the input Febuxostat used herein is selected from the group consisting of but not limited to crystalline or amorphous form or any solvate.

In an embodiment, Febuxostat crystal is dissolved in an ester solvent such as ethyl acetate at 50-60° C. and cooled to room temperature. The obtained solution is optionally filtered to remove any undissolved particulate, and the clear solution is subjected to spray drying in a buchi mini spray dryer (Model B-290) to obtain crystalline Febuxostat Form-M₁.

Another aspect of the present invention provides an improved process for the preparation of crystalline Form-K of Febuxostat comprising the steps of:
a) dissolving Febuxostat in an ester solvent,
b) evaporating the solvent by agitated thin film dryer (ATFD), and
c) isolating Febuxostat crystalline Form-K.

In one embodiment, the ester solvent used herein is Ethyl acetate.

In another embodiment, the input Febuxostat used herein is selected from the group consisting of but not limited to a crystalline or amorphous form or any solvate.

In an alternative embodiment, Febuxostat is dissolved in an ester solvent such as ethyl acetate at 50-60° C. and cooled to room temperature. The obtained solution is optionally filtered to remove any undissolved particulate, and the clear solution is evaporated on agitated thin film dryer (ATFD) instrument, at 50° C. under reduced pressure (120 mm-Hg) to obtain Febuxostat Form-K.

Another aspect of the present invention provides an improved process for the preparation of crystalline Form-K of Febuxostat comprising the steps of:
a) dissolving Febuxostat in an ester solvent at 50-80° C.,
b) removing the solvent at same temperature, and
c) isolating Febuxostat crystalline Form-K.

In one embodiment, the ester solvent used herein is Ethyl acetate.

In another embodiment, the solvent removed in step b is at a temperature of 70-80° C.

In another embodiment, the input Febuxostat used herein is selected from the group consisting of but not limited to a crystalline or amorphous form or any solvate.

In an alternative embodiment, Febuxostat is dissolved in an ester solvent such as ethyl acetate at 50-60° C., and cooled to room temperature (25-30° C.). The resulting solution is filtered through hyflow to remove any undissolved particulate and the solution was heated to 70-80° C. The solvent was distilled out at the same temperature under reduced pressure to obtain Febuxostat Form-K.

Yet another aspect of the present invention provides an improved process for the preparation of crystalline Form-K of Febuxostat comprising the steps of:
a) dissolving Febuxostat in an ester solvent
b) removing the solvent,
c) adding hydrocarbon solvent, and
d) isolating Febuxostat crystalline Form-K.

In one embodiment, the ester solvent used herein is Ethyl acetate.

In another embodiment, the solvent removed in step b is at a temperature of 70-80° C.

In another embodiment, hydrocarbon solvents used herein are selected from cyclohexane and toluene.

In another embodiment, the input Febuxostat used herein is selected from the group consisting of but not limited to a crystalline or amorphous form or any solvate.

In an alternative embodiment, Febuxostat is dissolved in an ester solvent such as ethyl acetate at 50-60° C., and cooled to room temperature (25-30° C.). The resulting solution is filtered through hyflow to remove any undissolved particulate and the solution is heated to 70-80° C. The solvent is distilled out at the same temperature under reduced pressure and hydrocarbon solvents such as toluene or cyclohexane are added to the residue. The resulting is slurred, filtered and dried to obtain Febuxostat Form-K.

Yet another aspect of the present invention provides an improved process for the preparation of crystalline Form-K of Febuxostat comprising the steps of:
a) dissolving Febuxostat in an ester solvent at 50-80° C.,
b) filtering the reaction mass,
c) maintaining the filtrate for 30-120 minutes at same temperature,
d) removing the solvent,
e) adding hydrocarbon solvent, and
f) isolating Febuxostat crystalline Form-K.

In one embodiment, the ester solvent used herein is Ethyl acetate.

In another embodiment, the reaction mass is filtered to separate any undissolved particulate.

In another embodiment, the filtrate is maintained for 30-120 minutes, preferably 60-90 minutes at 50-80° C.

In another embodiment, at step d, the solvent is removed at a temperature of 30-70° C. under reduced pressure.

In another embodiment, hydrocarbon solvent used herein is selected from cyclohexane and toluene.

In another embodiment, the input Febuxostat used herein is selected from the group consisting of but not limited to a crystalline or amorphous form or any solvate.

Yet another aspect of the present invention provides an improved process for the preparation of crystalline Form-K of Febuxostat comprising the steps of:
a) dissolving Febuxostat in one or more organic solvents,
b) adding a base to the reaction mixture,
c) removing the solvent,
d) adding water and neutralizing with acid,
e) adding one or more ester solvents,
f) optionally adding hydrocarbon solvent, and
g) isolating Febuxostat crystalline Form-K.

In one embodiment, the organic solvent is selected from ethanol, methanol, isopropanol, tetrahydrofuran or mixtures thereof.

In another embodiment, the base is selected from alkali or alkali earth metal hydroxides like sodium hydroxide or potassium hydroxide, preferably sodium hydroxide.

In another embodiment, the acid used in this invention for the neutralization is selected from acetic acid, formic acid, hydrochloric acid, sulfuric acid or phosphoric acid.

In another embodiment, the ester solvent used herein is Ethyl acetate.

In another embodiment, hydrocarbon solvents used herein are selected from cyclohexane or toluene.

In one more embodiment, the input Febuxostat used herein is selected from the group consisting of but not limited to a crystalline or amorphous form or any solvate.

In an alternative embodiment, Febuxostat is dissolved in organic solvents such as ethanol and tetrahydrofuran at 25-30° C. To this, a base such as sodium hydroxide is added and the solvent is distilled out at 60-70° C. The reaction mass is cooled to 25-30° C. and water is added. This is neutralized with hydrochloric acid and an ester solvent such as ethyl acetate is added. The reaction mass is completely distilled out at 75-80° C. under reduced pressure and cooled to 25-30° C. To the solid mass, cyclohexane is added and filtered. The resulting solid is dried to obtain Febuxostat crystalline Form-K.

Yet another aspect of the present invention provides an improved process for the preparation of crystalline Form-K of Febuxostat comprising the steps of:
a) dissolving alkyl ester of Febuxostat in one or more organic solvents,
b) adding a base to the reaction mixture,
c) removing the solvent,
d) adding water and neutralizing with acid,
e) adding one or more ester solvents,
f) optionally adding hydrocarbon solvent, and
g) isolating Febuxostat crystalline Form-K.

In one embodiment, the alkyl ester of Febuxostat is selected from methyl or ethyl ester, preferably ethyl ester.

In another embodiment, the organic solvent is selected from ethanol, methanol, isopropanol, tetrahydrofuran or mixtures thereof.

In another embodiment, the base is selected from alkali or alkali earth metal hydroxides like sodium hydroxide or potassium hydroxide, preferably sodium hydroxide.

In another embodiment, the acid used in this invention for the neutralization is selected from acetic acid, formic acid, hydrochloric acid, sulfuric acid and phosphoric acid.

In another embodiment, the ester solvent used herein is Ethyl acetate.

In another embodiment, hydrocarbon solvents used herein are selected from cyclohexane and toluene.

In an alternative embodiment, ethyl ester of Febuxostat is dissolved in organic solvents such as ethanol and tetrahydrofuran at 25-30° C. To this, a base such as sodium hydroxide is added and the solvent is distilled out at 60-70° C. The reaction mass is cooled to 25-30° C. and water is added. This is neutralized with hydrochloric acid and an ester solvent like ethyl acetate is added. The reaction mass is completely distilled out at 75-80° C. under reduced pressure and cooled to 25-30° C. To the solid mass, cyclohexane is added and filtered. The resulting solid is dried to obtain Febuxostat crystalline Form-K.

Yet another aspect of the present invention provides an improved process for the preparation of crystalline Form-K of Febuxostat comprising the steps of:
a) dissolving Febuxostat in an ester solvent,
b) partially removing the solvent,
c) adding hydrocarbon solvent, and
d) isolating Febuxostat crystalline Form-K.

In one embodiment, the ester solvent used herein is Ethyl acetate.

In another embodiment, hydrocarbon solvents used herein are selected from cyclohexane and toluene.

In another embodiment, the input Febuxostat used herein is selected from the group consisting of but not limited to a crystalline or amorphous form or any solvate.

Yet another aspect of the present invention provides an improved process for the preparation of crystalline Form-K of Febuxostat comprising the steps of:
a) dissolving Febuxostat in a mixture of ester and hydrocarbon solvent,
b) partially removing the solvent, and
c) isolating Febuxostat crystalline Form-K.

In one embodiment, the ester solvent used herein is Ethyl acetate and hydrocarbon solvent used herein is selected from cyclohexane and toluene. The preferable mixture of solvents is a mixture of ethyl acetate and toluene.

In another embodiment, at step b, 30-70%, preferably 40-55% of solvent is removed at a temperature of 50-100° C., preferably at atmospheric pressure.

In another embodiment, Febuxostat crystalline Form K is isolated by cooling the reaction temperature to 10-40° C., preferably 20-35° C. followed by filtration.

In another embodiment, the input Febuxostat used herein is selected from the group consisting of but not limited to a crystalline or amorphous form or any solvate.

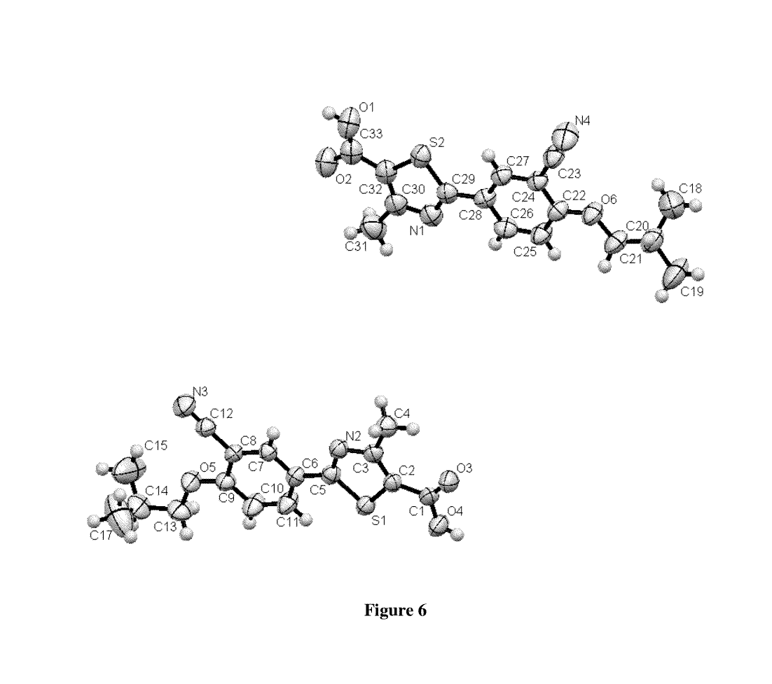

Febuxostat Form K is characterized by a single crystal X-ray diffraction pattern as shown in FIG. 6. The crystallographic data and atomic coordinates are incorporated respectively in Table 1 and Table 2.

TABLE 1
Crystallographic Data of Febuxostat Form
K from single crystal X-ray diffraction.

| Crystal system | Triclinic |
|---|---|
| Lattice type | Primitive |
| Space group | P-1 |
| Lattice parameters | a = 7.3660(15)Å |
| | b = 15.403(3)Å |
| | c = 15.721(3)Å |
| | α = 90.85(3)° |
| | β = 96.38(3)° |
| | γ = 98.75(3)° |
| | V = 1751.1(6)Å³ |
| Reflections (I > 2 σ) | 4048 |
| R-values: R, Rw. | 0.0777, 0.1626 |

TABLE 2
Atomic coordinates of Febuxostat Form K
obtained from single crystal X-ray diffraction.

| | Coordinates | | | |
|---|---|---|---|---|
| Atom | x | y | z | Ueq |
| S1 | 0.37859(14) | 0.06001(6) | 0.76398(6) | 0.0599 |
| S2 | 0.11124(14) | 0.87630(7) | 0.25622(7) | 0.0672 |
| O1 | 0.0449(5) | 0.9901(2) | 0.3948(2) | 0.0961 |
| O2 | 0.0308(5) | 0.8924(2) | 0.4972(2) | 0.0995 |
| O3 | 0.4619(4) | 0.1019(2) | 0.52581(18) | 0.0779 |
| O4 | 0.4517(5) | −0.0160(2) | 0.60628(19) | 0.0804 |
| O5 | 0.2721(4) | 0.20255(17) | 1.15913(17) | 0.0769 |
| O6 | 0.2427(4) | 0.65396(16) | −0.09539(19) | 0.0729 |
| N1 | 0.1411(5) | 0.7189(2) | 0.2993(2) | 0.0708 |
| N2 | 0.3579(4) | 0.2249(2) | 0.76064(19) | 0.0549 |
| N3 | 0.2567(6) | 0.4085(2) | 1.0843(3) | 0.0868 |
| N4 | 0.2208(7) | 0.8748(3) | −0.1236(3) | 0.1091 |
| C1 | 0.4420(5) | 0.0666(3) | 0.5940(3) | 0.0588 |
| C2 | 0.4022(5) | 0.1134(2) | 0.6696(2) | 0.0537 |
| C3 | 0.3871(5) | 0.2005(3) | 0.6801(2) | 0.0554 |
| C4 | 0.3986(6) | 0.2695(3) | 0.6138(3) | 0.0728 |
| C5 | 0.3504(5) | 0.1573(2) | 0.8113(2) | 0.0549 |
| C6 | 0.3215(5) | 0.1651(2) | 0.9021(2) | 0.0539 |
| C7 | 0.3092(5) | 0.2466(2) | 0.9363(2) | 0.052 |
| C8 | 0.2890(5) | 0.2566(2) | 1.0228(2) | 0.0519 |
| C9 | 0.2837(6) | 0.1845(2) | 1.0765(2) | 0.0602 |
| C10 | 0.2909(7) | 0.1027(3) | 1.0403(3) | 0.0818 |
| C11 | 0.3104(6) | 0.0934(3) | 0.9550(3) | 0.0749 |
| C12 | 0.2721(6) | 0.3410(3) | 1.0582(2) | 0.0606 |
| C13 | 0.2752(7) | 0.1332(3) | 1.2197(3) | 0.0868 |
| C14 | 0.2871(8) | 0.1747(4) | 1.3081(3) | 0.0975 |
| C15 | 0.1266(12) | 0.2194(5) | 1.3184(4) | 0.1854 |
| C17 | 0.4701(12) | 0.2329(5) | 1.3304(4) | 0.1826 |
| C18 | 0.1187(10) | 0.5774(4) | −0.2664(4) | 0.1467 |
| C19 | 0.3108(10) | 0.4601(4) | −0.2265(4) | 0.1361 |
| C20 | 0.2784(9) | 0.5544(3) | −0.2078(4) | 0.0916 |
| C21 | 0.2471(7) | 0.5621(3) | −0.1152(3) | 0.0797 |
| C22 | 0.2208(5) | 0.6774(2) | −0.0149(3) | 0.0618 |
| C23 | 0.2120(7) | 0.8251(3) | −0.0708(3) | 0.0735 |
| C24 | 0.2042(5) | 0.7658(2) | −0.0014(3) | 0.0572 |
| C25 | 0.2138(6) | 0.6218(3) | 0.0537(3) | 0.0692 |
| C26 | 0.1907(6) | 0.6537(3) | 0.1328(3) | 0.0692 |
| C27 | 0.1792(5) | 0.7959(3) | 0.0797(3) | 0.0617 |
| C28 | 0.1718(5) | 0.7409(3) | 0.1480(3) | 0.059 |
| C29 | 0.1449(5) | 0.7708(3) | 0.2339(3) | 0.0613 |
| C30 | 0.1120(6) | 0.7622(3) | 0.3716(3) | 0.0748 |
| C31 | 0.1088(7) | 0.7132(4) | 0.4537(3) | 0.1013 |
| C32 | 0.0904(5) | 0.8488(3) | 0.3604(3) | 0.0699 |
| C33 | 0.0547(6) | 0.9132(4) | 0.4225(3) | 0.0736 |
| H1 | 0.023(8) | 1.0218(11) | 0.4336(11) | 0.1443 |
| H4 | 0.493(7) | −0.0369(9) | 0.5654(16) | 0.1208 |
| H4a | 0.386(4) | 0.2419(3) | 0.5579(3) | 0.1092 |
| H4b | 0.301(2) | 0.3039(12) | 0.6172(12) | 0.1092 |
| H4c | 0.5160(17) | 0.3070(12) | 0.6239(11) | 0.1092 |
| H7 | 0.3144(5) | 0.2950(2) | 0.9016(2) | 0.0624 |
| H10 | 0.2826(7) | 0.0536(3) | 1.0742(3) | 0.0978 |
| H11 | 0.3162(6) | 0.0380(3) | 0.9322(3) | 0.0897 |
| H13a | 0.166(3) | 0.0890(15) | 1.2083(4) | 0.104 |
| H13b | 0.379(3) | 0.1022(19) | 1.2145(5) | 0.104 |
| H14 | 0.2827(8) | 0.1267(4) | 1.3485(3) | 0.117 |
| H15a | 0.137(5) | 0.243(4) | 1.3759(15) | 0.278 |
| H15b | 0.124(6) | 0.266(3) | 1.279(4) | 0.278 |
| H15c | 0.0147(13) | 0.1780(13) | 1.307(5) | 0.278 |
| H17a | 0.468(4) | 0.2881(18) | 1.303(4) | 0.274 |
| H17b | 0.493(5) | 0.243(4) | 1.3913(6) | 0.274 |
| H17c | 0.5666(17) | 0.205(2) | 1.311(4) | 0.274 |
| H18a | 0.0075(16) | 0.540(2) | −0.256(2) | 0.22 |
| H18b | 0.105(5) | 0.6374(12) | −0.256(2) | 0.22 |
| H18c | 0.141(4) | 0.570(3) | −0.3249(4) | 0.22 |
| H19a | 0.411(5) | 0.4463(13) | −0.187(2) | 0.204 |
| H19b | 0.200(2) | 0.4198(5) | −0.220(3) | 0.204 |
| H19c | 0.341(7) | 0.4552(10) | −0.2839(12) | 0.204 |
| H20 | 0.394(8) | 0.599(4) | −0.226(4) | 0.15 |
| H21a | 0.347(3) | 0.5419(15) | −0.0791(7) | 0.096 |
| H21b | 0.131(2) | 0.5269(11) | −0.1059(14) | 0.096 |
| H25 | 0.2248(6) | 0.5629(3) | 0.0460(3) | 0.0827 |
| H26 | 0.1874(6) | 0.6157(3) | 0.1782(3) | 0.0831 |
| H27 | 0.1673(5) | 0.8545(3) | 0.0880(3) | 0.0739 |
| H31a | −0.0126(16) | 0.708(2) | 0.4718(13) | 0.151 |
| H31b | 0.140(5) | 0.6557(9) | 0.4445(7) | 0.151 |
| H31c | 0.197(4) | 0.7448(13) | 0.4971(7) | 0.151 |

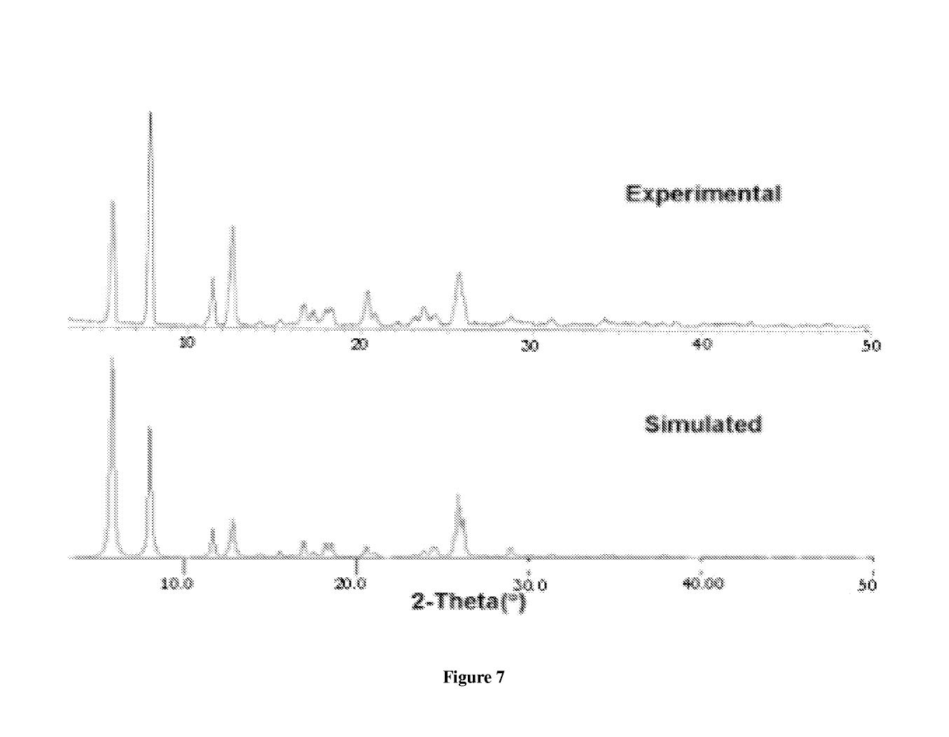

The experimental PXRD pattern was matched with the simulated PXRD pattern obtained from single crystal X-ray diffraction as shown in FIG. 7, which shows the phase purity of Form K.

A further aspect of the present invention is to provide packaging conditions for the stable crystalline Form-K of Febuxostat in a way to attain polymorphic stability, thereby increasing the shelf life of the product. According to the present invention the method for packaging crystalline Febuxostat Form-K comprises placing Febuxostat Form-K in a LDPE (low density polyethylene) bag under nitrogen atmosphere, placing the sealed bag in a Triple laminated aluminum liner bag with vacuumised nitrogen sealing, placing the above Triple laminated aluminum bag into the outer bag of the triple laminated aluminum bag and vacuumised nitrogen sealing and enclosing the triple laminated bag in closed HDPE (high density polyethylene) drums.

The invention is illustrated with the following examples, which are provided by way of illustration only and should not be construed to limit the scope of the invention.

### Experimental Procedure:

### Example—1

### Process for the Preparation of crystalline Febuxostat Form-M₁

5 g of Febuxostat Crystal A was dissolved in ethyl acetate (200 mL) at 50-60° C. and cooled to room temperature (25-30° C.). The resulting solution was filtered through hyflow to remove any undissolved particulate. The clear solution was subjected to spray drying in a Buchi mini spray dryer (Model B-290). The product was collected and dried at 50° C. under vacuum for 15 h. The resulted solid was identified as crystalline Febuxostat Form M₁.

### Example—2

### Process for the Preparation of crystalline Febuxostat Form-M₁

5 g of Febuxostat Crystal G was dissolved in ethyl acetate (200 mL) at 50-60° C. and cooled to room temperature (25-30° C.). The resulting solution was filtered through hyflow to remove any undissolved particulate. The clear solution was subjected to spray drying in a Buchi Mini Spray Dryer (B-290). The product was collected and dried at 50° C. under vacuum for 15h. The resulted solid was identified as crystalline febuxostat Form M₁.

### Example—3

### Process for the Preparation of Febuxostat Crystal Form-K

5 g of Febuxostat Crystal A was dissolved in ethyl acetate (200 mL) at 50-60° C. and cooled to room temperature (25-30° C.). The resulting solution was filtered through hyflow to remove any undissolved particulate. The clear solution was evaporated on agitated thin film dryer (ATFD) instrument, at 50° C. under reduced pressure (120 mm-Hg). The resulted solid was collected and identified as crystalline febuxostat Form K.

### Example—4

### Process for the Preparation of Febuxostat Crystal Form-K

5 g of Febuxostat Crystal G was dissolved in ethyl acetate (200 mL) at 50-60° C. and cooled to room temperature (25-30° C.). The resulting solution was filtered through hyflow to remove any undissolved particulate. The clear solution was evaporated on ATFD instrument at 65° C. under reduced pressure (120 mm-Hg). The resulted solid was collected and identified as crystalline febuxostat Form K.

### Example—5

### Process for the Preparation of Febuxostat Crystal Form-K

5 g of Febuxostat was dissolved in ethyl acetate (200 mL) at 70-80° C. and the solvent was removed on rotary evaporator at the same temperature under reduced pressure. The resulted solid was collected and identified as crystalline febuxostat Form K.

### Example—6

### Process for the Preparation of Febuxostat Crystal Form-K

10 g of Febuxostat was dissolved in ethyl acetate (150 mL) at 70-80° C. and the solvent was removed on rotary evaporator at the same temperature under reduced pressure. The resulting solid was collected and identified as crystalline febuxostat Form K.

### Example—7

### Process for the Preparation of Febuxostat Crystal Form-K

5 g of Febuxostat was dissolved in ethyl acetate (200 mL) at 50-60° C. and cooled to room temperature (25-30° C.). The resulting solution was filtered through hyflow to remove any undissolved particulate. The solution was heated to 70-80° C. and the solvent was distilled out at the same temperature under reduced pressure. The resulting solid was collected and identified as crystalline febuxostat Form K.

### Example—8

### Process for the Preparation of Febuxostat Crystal Form-K

10 g of Febuxostat was dissolved in ethyl acetate (250 mL) at 50-60° C. and cooled to room temperature (25-30° C.). The resulting solution was filtered through hyflow to remove any undissolved particulate. The solution was heated to 70-80° C. and the solvent was distilled out at the same temperature under reduced pressure. To the resulting solid mass 100 mL of toluene was added, slurried for 2 hours, filtered and dried over night at 50° C. under reduced pressure. The dry solid was identified as crystalline febuxostat Form K.

### Example—9

### Process for the Preparation of Febuxostat Crystal Form-K

10 g of Febuxostat was dissolved in ethyl acetate (250 mL) at 50-60° C., cooled to room temperature (25-30° C.). The resulting solution was filtered through hyflow to remove any undissolved particulate. The solution was heated to 70-80° C. and the solvent was distilled out at the same temperature under reduced pressure. To the resulting solid mass 100 mL of cyclohexane was added, slurried for 2 hours, filtered and dried over night at 50° C. under reduced pressure. The dry solid was identified as crystalline febuxostat Form K.

### Example—10

### Process for the Preparation of Febuxostat Crystal Form-K

10 g of Febuxostat was dissolved in ethyl acetate (250 mL) at 50-80° C. The resulting solution was filtered and maintained for 60-90 minutes at 50-80° C. The solvent was distilled out at 40-60° C. under reduced pressure. To the resulting solid mass 100 mL of cyclohexane was added and maintained for 2 hours. The reaction mass is centrifuged and the wet material is dried at 60° C. under reduced pressure. The dry solid was identified as crystalline Febuxostat Form K.

### Example—11

### Process for the Preparation of Febuxostat Crystal Form-K

5 g of Febuxostat was suspended in ethyl acetate (80 mL) at 25-30° C. and heated to 75-80° C. to obtain clear solution. The clear solution was distilled at 75-80° C. under reduced pressure to remove 60 mL of solvent. To the resulting suspension, cyclohexane (50 mL) was added at 75-80° C. and stirred for 10-15 minutes at 75-80° C. The reaction mass was further distilled to half of its volume at 70-80° C. under reduced pressure and filtered at 75-80° C. The product was isolated and dried at 60° C. under vacuum for 12-15 h. The solid obtained was identified as crystalline Febuxostat Form K.

### Example—12

### Process for the Preparation of Febuxostat Crystal Form-K

20 g of Febuxostat was suspended in EtOH (160 mL) and THF (220 mL) at 25-30° C. and stirred for 10-15 min to obtain clear solution. To this clear solution, aqueous NaOH solution (1N, 108 mL) was added slowly at 25-30° C., the temperature raised to 60° C. and the reaction mass stirred at 60° C. for 1 h. The solvent was distilled out completely under reduced pressure at 60-70° C. The reaction mass was cooled to 25-30° C., water (100 mL) was added and the resulting reaction mass was neutralized with IN HCl. Then ethyl acetate (400 mL) was added to the reaction mass and stirred for 10-15 minutes at 25-30° C. The aqueous layer was extracted using ethyl acetate (100 mL) and dried over anhydrous Na₂SO₄. The reaction mass was distilled out completely at 75-80° C. under reduced pressure. The solid obtained was identified as crystalline Febuxostat Form K.

### Example—13

### Process for the Preparation of Febuxostat Crystal Form-K

20 g of Febuxostat was suspended in EtOH (160 mL) and THF (220 mL) at 25-30° C. and stirred for 10-15 min to obtain a clear solution. To this clear solution, aqueous NaOH solution (1N, 108 mL) was added slowly at 25-30° C., the temperature was raised to 60° C. and the reaction mass was stirred at 60° C. for 1 h. The solvent was distilled out completely under reduced pressure at 60-70° C. The reaction mass was cooled to 25-30° C., water (100 mL) was added and the resulting reaction mass was neutralized with IN HCl. Then ethyl acetate (400 mL) was added to the reaction mass and stirred for 10-15 minutes at 25-30° C. The aqueous layer was extracted using ethyl acetate (100 mL) and dried over anhydrous Na₂SO₄ The reaction mass was distilled out completely at 75-80° C. under reduced pressure and cooled to 25-30° C. To the resulting solid mass, cyclohexane (100 mL) was added, slurried for 2 hours at 25-30° C. and filtered. The isolated solid was dried at 60° C. under vacuum for 12-15h. The product obtained was identified as crystalline Febuxostat Form K.

### Example—14

### Process for the Preparation of Febuxostat Crystal Form-K

20 g of ethyl ester of Febuxostat [ethyl 2-(3-cyano-4-isobutyloxyphenyl)-4-methyl-5-thiazolecarboxylate] was suspended in EtOH (160 mL) and THF (220 mL) at 25-30° C. and stirred for 10-15 min to obtain a clear solution. To this clear solution, aqueous NaOH solution (1N, 108 mL) was added slowly at 25-30° C., the temperature was raised to 60° C. and the reaction mass was stirred at 60° C. for 1h. The solvent was distilled out completely under reduced pressure at 60-70° C. The reaction mass was cooled to 25-30° C., water (100 mL) was added and the resulting reaction mass was neutralized with IN HCl. Then ethyl acetate (400 mL) was added to the reaction mass and stirred for 10-15 minutes at 25-30° C. The aqueous layer was extracted using ethyl acetate (100 mL) and dried over anhydrous Na₂SO₄. The reaction mass was distilled out completely at 75-80° C. under reduced pressure and cooled to 25-30° C. To the resulting solid mass, cyclohexane (100 mL) was added, slurried for 2 hours at 25-30° C. and filtered. The isolated solid was dried at 60° C. under vacuum for 12-15 h. The product obtained was identified as crystalline Febuxostat Form K.

### Example—15

### Process for the Preparation of Febuxostat Crystal Form-K

50 g of Febuxostat was suspended in ethyl acetate (1250 mL) at 25-30° C. and heated to 75-80° C. to obtain clear solution. The resulting solution was refluxed for 1 h and then cooled to 60° C. The solvent was distilled at 60-65° C. under reduced pressure to leave about 400-500 mL of solvent. To the resulting suspension, cyclohexane (500 mL) was added at 35° C. and stirred for 1 h at 25-30° C. and filtered. The product was isolated, dried at 60° C. under vacuum and unloaded under controlled humidity 40±10% RH and packed. The solid obtained was identified as crystalline Febuxostat Form K.

### Example—16

### Process for the Preparation of Febuxostat Crystal Form-K

50 g of Febuxostat was suspended in ethyl acetate (1000 mL) and toluene (500 mL) at 25-30° C. and heated to 75-80° C. to obtain clear solution. The resulting solution was refluxed for 1 h. The solution was distilled at 80-90° C. to leave about 700 mL of solution. The clear solution was cooled to 70° C. in 30 minutes and maintained at 70° C. for an additional 30 minutes. The hazy solution was further cooled to 25-30° C. in about 1 h and maintain at 25-30° C. for 30 minutes. The slurry was filtered, dried at 60° C. under vacuum and unloaded under controlled humidity 40±10% RH and packed. The solid obtained was identified as crystalline Febuxostat Form K.

### Packaging:

Febuxostat Form-K was packed in a LDPE bag under nitrogen atmosphere, twisted and tied with a plastic fastener. It was inserted in a triple laminated aluminum liner bag with vacuumised nitrogen sealing. Both these bags were then put into the outer bag of a triple laminated aluminum bag with vacuumised nitrogen sealing. The poly bags were further packed in HDPE drums, closed with plastic lids having a rubber gasket, followed by a locking ring and a metal seal and labeled.

We claim:

1. A process for the preparation of crystalline Form-K of Febuxostat comprising the steps of:
a) dissolving Febuxostat in an ester solvent,
b) removing the solvent,
c) adding hydrocarbon solvent, and
d) isolating Febuxostat crystalline Form-K.

2. The process according to claim 1, wherein the ester solvent is ethyl acetate.

3. The process according to claim 1, wherein the hydrocarbon solvent is selected from cyclohexane or toluene.

4. A process for the preparation of crystalline Form-K of Febuxostat comprising the steps of:
a) dissolving Febuxostat in an ester solvent at 50-80° C.,
b) filtering the reaction mass,
c) maintaining the filtrate for 30-120 minutes at same temperature,
d) removing the solvent,
e) adding hydrocarbon solvent, and
f) isolating Febuxostat crystalline Form-K.

5. The process according to claim 4, wherein the ester solvent is ethyl acetate.

6. The process according to claim 4, wherein the hydrocarbon solvent is selected from cyclohexane or toluene.

7. A process for the preparation of crystalline Form-K of Febuxostat comprising the steps of:
a) dissolving Febuxostat in a mixture of an ester and hydrocarbon solvent,
b) partially removing the solvent, and
c) isolating Febuxostat crystalline Form-K.

8. The process according to claim 7, wherein ester solvent is ethyl acetate.

9. The process according to claim 7, wherein hydrocarbon solvent is selected from cyclohexane or toluene.

10. The process according to claim 7, wherein the mixture of solvents is a mixture of ethyl acetate and toluene.

11. A process for the preparation of crystalline Form-K of Febuxostat comprising the steps of:
a) dissolving Febuxostat in an ester solvent,
b) evaporating the solvent by agitated thin film dryer (ATFD), and
c) isolating Febuxostat crystalline Form-K.

12. The process according to claim 11, wherein the ester solvent is ethyl acetate.

13. A process for the preparation of pure crystalline Form-K of Febuxostat comprising the steps of:
a) dissolving Febuxostat in an ester solvent at 50-80° C.,
b) removing the solvent and
c) isolating Febuxostat crystalline Form-K.

14. The process according to claim 13, wherein the ester solvent is ethyl acetate.

15. A process for the preparation of crystalline Form-K of Febuxostat comprising the steps of:
a) dissolving Febuxostat in one or more organic solvents,
b) adding a base to the reaction mixture,
c) removing the solvent,
d) adding water and neutralizing with acid,
e) adding one or more ester solvents,
f) optionally adding hydrocarbon solvent, and
g) isolating Febuxostat crystalline Form-K.

16. The process according to claim 15, wherein the organic solvent is selected from ethanol, methanol, isopropanol, tetrahydrofuran or mixtures thereof.

17. The process according to claim 15, wherein the base is selected from alkali or alkali earth metal hydroxides.

18. The process according to claim 15, wherein the acid used for the neutralization is selected from acetic acid, formic acid, hydrochloric acid, sulfuric acid or phosphoric acid.

19. The process according to claim 15, wherein the ester solvent is ethyl acetate.

20. The process according to claim 15, wherein the hydrocarbon solvent is selected from cyclohexane or toluene.

21. A process for the preparation of crystalline Form-K of Febuxostat comprising the steps of:
a) dissolving alkyl ester of Febuxostat in one or more organic solvents,
b) adding a base to the reaction mixture,
c) removing the solvent,
d) adding water and neutralizing with acid,
e) adding one or more ester solvents,
f) optionally adding hydrocarbon solvent, and
g) isolating Febuxostat crystalline Form-K.

22. The process according to claim 21, wherein the alkyl ester of Febuxostat is selected from methyl or ethyl ester.

23. The process according to claim 21, wherein the organic solvent is selected from ethanol, methanol, isopropanol, tetrahydrofuran or mixtures thereof.

24. The process according to claim 21, wherein the base is selected from alkali or alkali earth metal hydroxides.

25. The process according to claim 21, wherein the acid used for the neutralization is selected from acetic acid, formic acid, hydrochloric acid, sulfuric acid or phosphoric acid.

26. The process according to claim 21, wherein the ester solvent is ethyl acetate.

27. The process according to claim 21, wherein the hydrocarbon solvent is selected from cyclohexane or toluene.

28. An improved process for the preparation of crystalline Form-K of Febuxostat comprising the steps of:
a) dissolving Febuxostat in an ester solvent,
b) partially removing the solvent,
c) adding a hydrocarbon solvent, and
d) isolating Febuxostat crystalline Form-K.

29. The process according to claim 28, wherein the ester solvent is ethyl acetate.

30. The process according to claim 28, wherein the hydrocarbon solvent is selected from cyclohexane or toluene.

31. A method of packaging crystalline Febuxostat Form-K comprising:
a) placing Febuxostat Form-K in a LDPE (low density polyethylene) bag under nitrogen atmosphere,
b) placing the sealed bag in a triple laminated aluminum liner bag with vacuumised nitrogen sealing,
c) placing the above bag into the outer bag of a triple laminated aluminum bag with vacuumised nitrogen sealing, and
d) enclosing the triple laminated bag in closed HDPE (high density polyethylene) drums.

32. Crystalline Form M, of Febuxostat.

33. Crystalline Form M, according to claim 32, wherein Form M, of Febuxostat is characterized by the powder X-ray diffraction having characteristic peaks 6.13, 9.06, 12.29, 17.44 and 25.81±0.2°2θ.

34. Crystalline Form M, according to claim 32, wherein Form M, of Febuxostat is characterized by Powder X-ray diffraction as depicted in FIG. 1.

35. Crystalline Form M, according to claim 32, wherein Form M, of Febuxostat is characterized by the DSC thermogram as depicted in FIG. 2.

36. A process for the preparation of Febuxostat crystalline Form-M, comprising the steps of:
a) dissolving Febuxostat in an ester solvent,
b) spray drying the clear solution, and
c) isolating the crystalline Form-M₁.

37. The process according to claim 36, wherein the ester solvent is ethyl acetate.
